(19) United States
(12) Patent Application Publication
(10) Pub. No.: US 2015/0182376 A1
(43) Pub. Date: Jul. 2, 2015

(54) Therapeutic Gloves with Heating Pads

(71) Applicant: Peggy Fortner, Newport, IN (US)

(72) Inventor: Peggy Fortner, Newport, IN (US)

(21) Appl. No.: 14/588,184

(22) Filed: Dec. 31, 2014

Related U.S. Application Data
(60) Provisional application No. 61/922,213, filed on Dec. 31, 2013.

(51) Int. Cl. A61F 7/08; A41D 19/00; A41D 19/01

(52) U.S. Cl. CPC A61F 7/08; A41D 19/01; A41D 19/0024

(57) ABSTRACT

Disclosed is a therapeutic glove with a removable heating pad. In one embodiment, the glove includes a thumb compartment and a finger and palm compartment. The finger and palm compartment includes a pocket on the top side and on the palm side thereof. Each pocket is elongated and spans between the front end and the wrist portion of the glove. Additionally, each pocket is adapted to enclose a heating pad therein, wherein the heating pad is shaped and dimensioned similarly to the pocket. Accordingly, the heating pad covers a substantial portion of the user's hand. The heating pad is reusable, and can transfer heat to the user's hand when placed in the pocket.

### CROSS REFERENCE TO RELATED APPLICATION

This application claims the benefit of U.S. Provisional Application No. 61/922,213 filed on Dec. 31, 2013. The above identified patent application is herein incorporated by reference in its entirety to provide continuity of disclosure.

### BACKGROUND OF THE INVENTION

1. Field of the Invention

The present invention relates to a therapeutic glove. More specifically, the present invention pertains to an improved therapeutic glove with heating pads removably secured thereto. The present invention is adapted to apply heat to the user's hands to alleviate joint and muscle pains in the hand.

The hand and wrist have multiple small joints that work together to produce motion, including the fine motion needed to conduct daily activities. When the joints are affected by arthritis or other joint and muscle conditions, activities of daily living can be difficult. Arthritis can occur in many areas of the hand and wrist and can have more than one cause. Over time, if the arthritis is not treated, the bones that make up the joint can lose their normal shape. This causes more pain and further limits motion. Thus, it is important to properly treat arthritis.

Nonsurgical treatment options for arthritis of the hand and wrist include medication, splinting, and injections. Additionally, many individuals apply heat to increase blood flow, enhance flexibility, provide carpel tunnel relief, and decrease arthritic pains. Traditionally, individuals utilize a heat pack and place it on their hands. Heat packs, however, generally do not include any fastening means to secure to the individual's hand. Accordingly, individuals must place the heat pack on one hand and hold it down with their other hand, leaving them without a free hand. Alternatively, individuals utilize a wrap or a strap to secure a heat pack to their hand. Wraps and straps, however, are not adapted to be worn on the hand. Therefore, a device that can provide heat therapy to the hand, and that can be worn during daily activities is desired.

2. Description of the Prior Art

Devices have been disclosed in the prior art that claim heated gloves. These include devices that have been patented and published in patent application publications. Some of these devices disclose hand warming gloves that include an externally accessible pocket for receiving a heat pack. Other devices disclose a glove with a sealed inner pocket that holds a hand warmer. These devices, however, do not disclose therapeutic gloves with an upper and a lower pockets for holding hand warmers therein. The foregoing is a list of devices deemed most relevant to the present disclosure, which are herein described for the purposes of highlighting and differentiating the unique aspects of the present invention, and further highlighting the drawbacks existing in the prior art.

Specifically, U.S. Pat. No. 7,251,837 to Horton discloses a hand warming apparatus that comprises a wrist wrap and a heating assembly attached thereto. The heating assembly is disposed on the portion of the wrist wrap that corresponds to the inner wrist of the user. Additionally, the heating assembly comprises wiring so as to distribute heat throughout the wrist wrap. Horton, therefore, does not disclose heating pads that are positioned on the back of the user's hand and on the user's palm.

Similarly, U.S. Pat. No. 4,535,482 to Spector discloses a heated glove having a heating assembly that is located on the top side of the glove. The heating assembly is adapted to provide heat to the back of the user's hand and to each of the user's fingers. In contrast, the present invention comprises a heating pad that spans the back of the user's hand and the user's palm to provide heat to the joints and muscles of the user's hand. The heating pad is substantially rectangular in shape and is elongated so as to span a substantial portion of the user's hand.

U.S. Patent Application Publication Number 2011/0162127 to Allen and U.S. Pat. No. 6,141,801 to Helenick disclose gloves with a hand warmer compartment. The hand warmer compartment is located on the top portion of the glove, wherein the top portion corresponds with the back of the user's hand. The hand warmer compartment, however, does not extend toward the user's fingers. Additionally, Allen does not disclose a hand warmer compartment on the palm portion of the glove.

Finally, U.S. Pat. No. 7,043,768 to Gogarty discloses a thermal gel filled glove. The glove comprises an exterior layer, an interior layer, and a defined interior volume therein, wherein the interior volume is filled with thermal gel that can be heated. In contrast, the present invention comprises heating pads that can be removably inserted into the glove. Additionally, the present invention comprises a mitten. Gogarty discloses a glove with thermal gel integral therewith. Accordingly, Gogarty does not disclose heating pads that can be removably inserted into the palm portion and the top portion of the glove.

The devices disclosed in the prior art have several known drawbacks. These devices are limited in that they are designed to only heat the back of the user's hand. Alternatively, these devices require a user to seek a power source to supply power to actuate the heating assembly. The present invention overcomes these limitations by disclosing a therapeutic glove with removable heating pads. The glove is substantially hand-shaped so that it can be comfortably worn by the user and remain in place without fastening means. In one embodiment, the glove comprises a mitten having a top portion and a palm portion, each of the top and palm portions having a pocket thereon. Each pocket is configured to secure a removable heating pad therein. In one embodiment, the heating pad comprises an elongated casing having gel material embedded therein. The heating pad can be heated in hot water or in a microwave. The heating pad is adapted to retain heat for an extended period of time, and is reusable.

It is therefore submitted that the present invention is substantially divergent in design elements from the prior art, and consequently it is clear that there is a need in the art for an improvement to heated gloves. In this regard, the instant invention substantially fulfills these needs.

### SUMMARY OF THE INVENTION

In view of the foregoing disadvantages inherent in the known types of heated gloves now present in the prior art, the present invention provides a new and improved therapeutic gloves with heating pads wherein the same can be utilized for applying heat to the wearer's hands and alleviating pain.

It is therefore an object of the invention to provide a new and improved therapeutic gloves with heating pads that has all of the advantages of the prior art and none of the disadvantages.

Another object of the present invention is to provide a new and improved therapeutic gloves with heating pads that is adapted to provide heat to the user's palm and the back of the user's hand.

Yet another object of the present invention is to provide a new and improved therapeutic gloves with heating pads that can conform to the contours of the user's hand and that can be worn during daily activities.

Still yet another object of the present invention is to provide a new and improved therapeutic gloves with heating pads wherein the heating pads can be removably inserted into the gloves.

Still yet another object of the present invention is to provide a new and improved therapeutic gloves with heating pads wherein the heating pads are reusable.

Still yet another object of the present invention is to provide a new and improved therapeutic gloves with heating pads wherein the device may be readily fabricated from materials that permit relative economy and are commensurate with durability.

Other objects, features, and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein the numeral annotations are provided throughout.

FIG. 1 shows a perspective view of the present invention.

FIG. 2 shows a cross sectional view of the present invention as worn.

### DETAILED DESCRIPTION OF THE INVENTION

References are made herein to the attached drawings. Like reference numerals are used throughout the drawings to depict like or similar elements of the therapeutic gloves with heating pads. For the purposes of presenting a brief and clear description of the present invention, the preferred embodiment will be discussed as used to apply heat to the wearer's hands and to alleviate pain. The figures are intended for representative purposes only and should not be considered to be limiting in any respect.

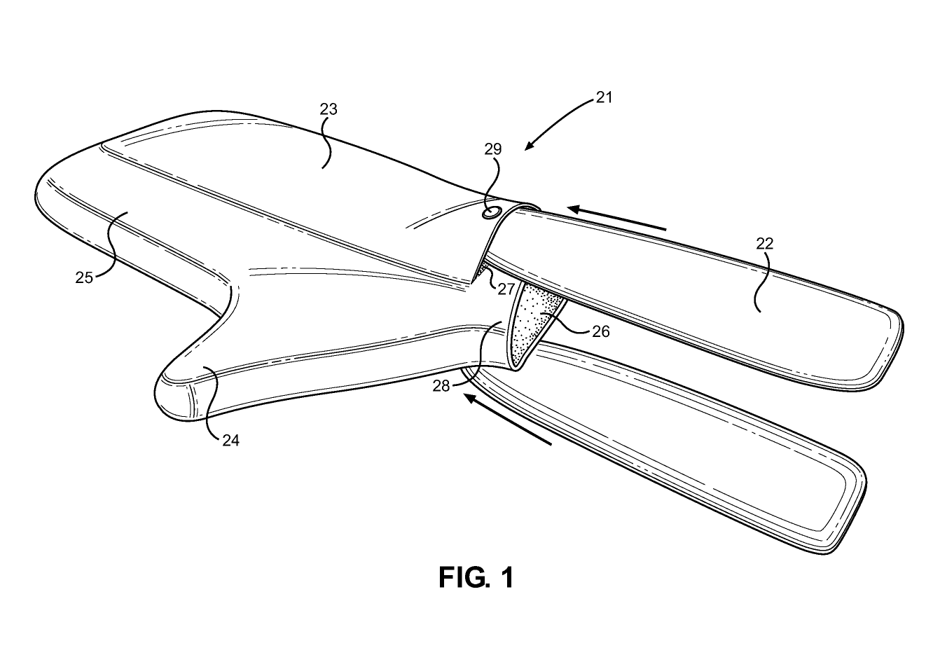

Referring now to FIG. 1, there is shown a perspective view of the present invention. In the illustrated embodiment, the present invention comprises a substantially hand-shaped mitten 21 having a thumb compartment 24, a finger and palm compartment 25, and an opening 26 at a wrist portion 28. The mitten 21 is composed of soft and plush fabric such as cotton or other suitable material known in the art for constructing gloves. Additionally, the mitten 21 may be composed of moisture-wicking fabric to draw sweat off the skin of the user's hand to the outside of the fabric. The mitten 21 further comprises a top side that corresponds with the back of a user's hand, and a palm side that corresponds with the palm of the user's hand.

Each of the top side and the palm side of the mitten 21 comprises a pocket 23 thereon. The pocket 23 is elongated and substantially rectangular in shape. The pocket 23 is sewn about the edges thereof, leaving one edge open to provide an opening 27 at the wrist portion 28 of the mitten 21. The opening 27 provides an access to the interior volume of the pocket 23. The interior volume of the pocket 23 is adapted to receive a heating pad 22 therein. The heating pad 22 is shaped and dimensioned so as to fit within the interior volume of the pocket 23. Accordingly, the heating pad 22 is generally rectangular in shape.

Preferably, the heating pad 22 fits snugly within the pocket 23 so it does not shift excessively during use. In some embodiments, the inside of the pocket 23 may be lined with a high friction material to prevent the heating pad 22 from slipping out of the pocket 23. The pocket 23 comprises a fastener 29 thereon to secure the heating pad 22 therein. In the illustrated embodiment, the fastener 29 comprises snap buttons. One snap button may be disposed near the open edge of the pocket 23. The snap button may be directly aligned with a mating snap button on the top side or the palm side of the mitten 21. Other fasteners such as hook and loop fasteners or buttons may be used in other embodiments.

The heating pad 22 comprises a casing that is completely sealed around the edges to prevent the gel material contained within its interior volume from leaking. The heating pad 22 is malleable, but substantially firm, and can be compressed against the user's features and molded thereto when deployed. This allows the heating pad 22 to contour around various parts of the hand, providing comfort to the user.

The gel material contained in the casing is preferably water-based gel that is capable of retaining heat for an extended period of time, and that can be reused. The heat pack 22 may be heated in boiling water or heated in a microwave. Preferably, the gel material comprises a non-toxic gel that can absorb a considerable amount of heat due to the high enthalpy of fusion of water. The gel material may comprise a family of gel materials, including hydroxyethyl cellulose or vinyl-coated silica gel. Alternatively, the gel material may comprise beaded gel or microwavable micro beads so as to increase the flexibility of the heat pack 22. The micro beads are adapted to absorb moisture from the air and release it when heated, delivering warmth to the user.

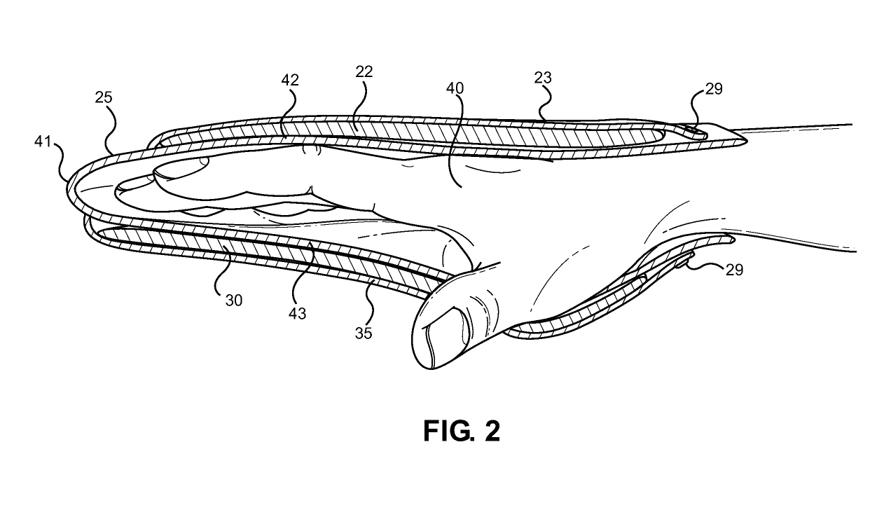

Referring now to FIG. 2, there is shown a cross sectional view of the present invention as worn. The mitten is worn so that the user's thumb is enclosed in the thumb compartment, and the user's fingers and palm 40 are enclosed in the finger and palm compartment 25. Additionally, the top side 42 corresponds with the back of the user's hand, and the palm side 43 corresponds with the user's palm. The top side 42 includes a pocket 23 thereon, wherein the pocket 23 spans between the front end 41 and the wrist portion of the mitten. The pocket 23 comprises a defined interior volume in which a heating pad 22 is placed. The heating pad 22 spans from the tip of the user's fingers to the user's wrist at the back of the user's hand, substantially covering the back of the user's hand. The heating pad 22 is removably secured within the pocket 23 via a snap button 29 disposed near the opening of the pocket 23.

Similarly, the palm side 43 comprises a pocket 35 thereon. The pocket 35 spans between the front end 41 and the wrist portion of the mitten. The pocket 35 comprises a defined interior volume in which a heating pad 30 is placed. The heating pad 30 spans from the tip of the user's fingers to the user's wrist at the user's palm, substantially covering the user's fingers and palm. The heating pad 30 is removably secured within the pocket 35 via a snap button 29 disposed near the opening of the pocket 35. It is contemplated, however, other embodiments of the present invention may comprise other fastening means such as hook and loop fasteners, and buttons, among others.

The heating pads 22, 30 are of sufficient length and width to cover a substantial portion of the user's hand. Additionally, the heating pads 22, 30 preferably comprise a low profile so as to reduce bulkiness. The heating pads 22, 30 can be heated in boiling water, in a microwave, or by other means. The heat from the heating pads 22, 30 can pass through the top side 42 and the palm side 43 of the mitten to provide heat therapy to the user's hand. In this way, the user's hand is not directly exposed to the heating pads 22, 30, which can help prevent the user from burning his or her hand. The heating pads 22, 30 can retain heat for an extended period of time, and can be reheated as desired by the user. The heat from the heating pads 22, 30 provides therapeutic benefits, including increase of blood flow and reduction of pain from arthritis or other joint conditions. The mitten may be worn during daily activities or during sleep.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above descriptions then, it is to be realized that the optimum dimensional relationships for the parts of the invention, to include variations in size, materials, shape, form, function, and manner of operation, assembly and use, are deemed readily apparent and obvious to one skilled in the art, and all equivalent relationships to those illustrated in the drawings and described in the specifications are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A therapeutic glove, comprising:
a mitten having a top side and a palm side;
wherein said top side corresponds with a back of a user's hand and said palm side corresponds with a user's palm;
each of said top side and said palm side having a pocket;
a heating pad removably inserted in said pocket.

2. The therapeutic glove of claim 1, wherein said mitten further comprises a thumb compartment and a finger and palm compartment.

3. The therapeutic glove of claim 2, wherein said pocket is disposed on said finger and palm compartment.

4. The therapeutic glove of claim 1, wherein said pocket spans between a front end of said mitten and a wrist portion of said mitten.

5. The therapeutic glove of claim 1, wherein said heating pad spans between a front end of said mitten and a wrist portion of said mitten.

6. The therapeutic glove of claim 5, wherein said heating pad is dimensioned to fit snugly in said pocket.

7. The therapeutic glove of claim 1, wherein said heating pad comprises a casing with a gel material therein;
wherein said heating pad is adapted to retain heat for an extended period of time.

8. The therapeutic glove of claim 1, wherein said pocket further comprises a fastener at an opening thereof.

9. The therapeutic glove of claim 8, wherein said fastener comprises a snap button.
